(19) United States
(12) Patent Application Publication
(10) Pub. No.: US 2019/0360020 A1
(43) Pub. Date: Nov. 28, 2019

(54) OIL-PHASE COMPOSITION FOR GENERATING WATER-IN-OIL LIQUID DROPS BY MEANS OF CENTRIFUGATION

(71) Applicant: GEEKGENE BIOTECHNOLOGY, INC., Beijing (CN)

(72) Inventors: Yanyi HUANG, Beijing (CN); Zitian CHEN, Beijing (CN); Fangli ZHANG, Beijing (CN); Peiyu LIAO, Beijing (CN)

(73) Assignee: Geekgene Biotechnology, Inc., Beijing (CN)

(21) Appl. No.: 16/309,093

(22) PCT Filed: May 25, 2017
(86) PCT No.: PCT/CN2017/085892

§ 371 Date: Aug. 15, 2019

(30) Foreign Application Priority Data
Jun. 12, 2016 (CN) 201610409462.8

(51) Int. Cl. C12Q 1/6806; B01F 17/00; B01F 17/54

(52) U.S. Cl. CPC C12Q 1/6806; B01F 17/0021; B01F 17/0071; B01F 17/0092

(57) ABSTRACT

An oil-phase composition for generating water-in-oil droplets by means of centrifugation, consisting of the following components: 7-15% (v/v) of a long-chain alkyl-containing silicon-oxygen chain nonionic surfactant and 0-10% of mineral oil, with the balance being diethylhexyl carbonate; or consisting of the following components: 85-95% (v/v) of a long-chain alkane ester and 5-15% (v/v) of a long-chain alkyl-containing silicon-oxygen chain nonionic surfactant; or consisting of silicone oil and a surfactant. Also provided is a method for generating water-in-oil droplets by means of centrifugation using the oil-phase composition as a second liquid.

### TECHNICAL FIELD

Examples of the present invention relate to an oil-phase composition for generating droplets, and a method for generating water-in-oil droplets by centrifugation using the oil-phase composition as a second liquid.

### BACKGROUND

At present, microfluidics is the commonly used method for realizing dropletization, but with limitations, like inability to achieve high throughput, and high demands on environmental cleanliness and cumbersome operation in preparation and use of microfluidic chips, etc.

### SUMMARY

Examples of the present invention provide an oil-phase composition consisting of the following components: 7%-15% (v/v) of a long-chain alkyl-containing silicon-oxygen chain nonionic surfactant, and 0%-10% (v/v) of mineral oil, with the balance being diethylhexyl carbonate.

According to one embodiment of the present invention, for example, in the above oil-phase composition, the long-chain alkyl-containing silicon-oxygen chain nonionic surfactant comprises cetyl polyethylene glycol/polypropylene glycol-10/1 dimethicone (CETYL PEG/PPG-10/1 DIMETHICONE) or a similar structure.

According to one embodiment of the present invention, for example, in the above oil-phase composition, the long-chain alkyl-containing silicon-oxygen chain nonionic surfactant is selected from at least one of the following surfactants: ABIL® WE09, ABIL® EM180, ABIL® EM90, BC2426(KCC Group), DIDW series from Kobo Products, Inc., and Silok® 2215, 2216 and 2216C from the Silok Chemical company.

Examples of the present invention provide an oil-phase composition consisting of the following components: 85%-95% (v/v) of a long-chain alkane ester, and 5%-15% (v/v) of a long-chain alkyl-containing silicon-oxygen chain nonionic surfactant.

According to one embodiment of the present invention, for example, in the above oil-phase composition, the long-chain alkane ester has 10 or more carbon atoms.

According to one embodiment of the present invention, for example, in the above oil-phase composition, the long-chain alkane ester has a freezing point of −10° C.-20° C.

According to one embodiment of the present invention, for example, in the above oil-phase composition, the long-chain alkane ester is at least one of isopropyl palmitate, butyl laurate, methyl laurate, ethyl laurate, and butyl stearate.

According to one embodiment of the present invention, for example, in the above oil-phase composition, the long-chain alkane ester is selected from at least one of methyl palmitate, ethyl palmitate, isopropyl palmitate, methyl laurate, ethyl laurate, propyl laurate, isoamyl laurate, butyl laurate, methyl oleate, ethyl oleate, glyceryl oleate, methyl stearate, ethyl stearate, vinyl stearate, butyl stearate, and glyceryl stearate.

According to one embodiment of the present invention, for example, in the above oil-phase composition, the long-chain alkyl-containing silicon-oxygen chain nonionic surfactant comprises CETYL PEG/PPG-10/1 DIMETHICONE or a similar structure.

According to one embodiment of the present invention, for example, in the above oil-phase composition, the long-chain alkyl-containing silicon-oxygen chain nonionic surfactant is selected from at least one of the following surfactants: ABIL® WE09, ABIL® EM180, ABIL® EM90, BC2426(KCC Group), DIDW series from Kobo Products, Inc., and Silok® 2215, 2216 and 2216C from the Silok Chemical company.

Examples of the present invention provide an oil-phase composition consisting of silicone oil and a surfactant, wherein the surfactant is selected from at least one of 5225C Formulation Aid, ES-5227 DM Formulation Aid, and ES-5612 and ES-5226 DM Formulation Aid.

According to one embodiment of the present invention, for example, in the above oil-phase composition, the silicone oil is 317667 silicone oil or 378321 silicone oil.

According to one embodiment of the present invention, for example, in the above oil-phase composition, when the surfactant is 5225C Formulation Aid, the content of the surfactant is 20%-50% (w/w), and the content of the silicone oil is 80%-50% (w/w); when the surfactant is ES-5227 DM Formulation Aid, the content of the surfactant is 10%-40% (w/w), and the content of the silicone oil is 90%-60% (w/w); when the surfactant is ES-5612, the content of the surfactant is 2%-15% (w/w), and the content of the silicone oil is 98%-85% (w/w); and when the surfactant is ES-5226 DM Formulation Aid, the content of the surfactant is 10%-30% (w/w), and the content of the silicone oil is 90%-70% (w/w).

Examples of the present invention provide a method for generating water-in-oil droplets by centrifugation using the oil-phase composition as a second liquid, comprising the steps of: adding to a dropletizing device a first liquid, adding to a collecting device the second liquid, and setting a rotational speed of an acceleration generating device, to generate the droplets, wherein the first liquid is in an amount of 5 μL-100 μL, the second liquid is in an amount of 300 μL-1500 μL, a vertical distance between a lower surface of the first liquid in the dropletizing device and an upper surface of the second liquid is controlled to be less than 1 cm to reduce a probability of breaking of the droplets when entering a liquid surface of the oil-phase composition, the dropletizing device comprises a porous plate for centrifugation with a pore diameter of 3 μm-10 μm and a centrifugal force of 5000 rcf-20000 rcf, which can generate uniform droplets with a diameter of 30 μm-200 μm, and the size of the droplets is mainly determined by the pore diameter and the centrifugal force of the porous plate for centrifugation.

### BRIEF DESCRIPTION OF THE DRAWINGS

In order to more clearly illustrate the technical solutions of the examples of the present invention, the drawings of the examples will be briefly described below. It is obvious that the drawings in the following description relate only to some examples of the present invention, and are not intended to limit the present invention.

FIG. 1 is a schematic diagram of a device for generating droplets by centrifugation.

FIG. 2 is a three phase diagram of an oil-phase composition consisting of diethylhexyl carbonate-ABIL WE09-mineral oil.

FIG. 3 is a bright field diagram of droplets generated by a Dolomite microfluidic chip (with a pore diameter of 100 μm).

FIG. 4 shows photos of droplets taken under the bright field (top) and the fluorescence microscope (bottom) after the PCR reaction of droplets generated by centrifugation.

### DETAILED DESCRIPTION OF THE INVENTION

In order to make the purpose, technical solutions and advantages of the examples of the present invention more clear, the technical solutions of the examples of the present invention will be clearly and completely described in the following with reference to the accompanying drawings of the examples of the present invention. It is apparent that the described examples are part, not all of the examples of the present invention. All other examples obtained by a person of ordinary skill in the art based on the described examples of the present invention without paying creative effort are within the protection scope of the present invention.

To break through the limitations of the microfluidic method, droplets can be generated by centrifugation. In centrifugation, the density of the oil phase is required to be less than that of water to enable the quick sinkage of the aqueous-phase droplets generated by the dropletizing device, so as to reduce the collision among droplets. In addition, the droplets need to possess good thermal stability (e.g., the PCR reaction requires 95° C.) and biocompatibility to ensure the occurrence of subsequent reactions. This correspondingly needs a new type of oil-phase formulations.

At present, there are three main types of oil phases for forming stable droplets based on microfluidic platforms: fluorocarbon oils, including FC 3283, F40, and FC70, etc.; hydrocarbon oils, including mineral oils, alkanes, etc.; and silicone oils. Surfactants are required to add into all three types of oil phases to stabilize the emulsion. However, silicone oils are not compatible with PDMS, and have high requirement on the material of the microfluidic chips, and there are few studies on the surfactants used together with silicone oils. The existing hydrocarbon oil formulations have good thermal stability, but there is still droplet fusion phenomenon at a high temperature of 95° C. Fluorocarbon oils are suitable for microfluid, have good stability and biocompatibility, but are not suitable for centrifugation because their density is much larger than water. In recent years, oil phase formulations combining mineral oil and surfactant are widely used in emulsion PCR and BEAMing PCR. However, inventors of the present invention have found that although such formulations can generate uniform aqueous-phase droplets as a continuous phase virtually without loss in microfluidic chips, and can also generate uniform and stable droplets by centrifugation, the thermal stability of the droplets generated by centrifugation cannot meet the temperature condition of the PCR reaction.

With regard to the above problem, examples of the present invention provide an oil-phase composition, which is suitable for centrifugation and also has good stability, ensuring the occurrence of subsequent reactions. The reagents used are low in cost and easy to obtain. The combination of the oil-phase composition of the examples of the present invention and centrifugation provides a faster and more convenient method with a higher throughput for generating droplets than microfluidics. The oil formulation can form a stable emulsion with a buffer containing salt ions and can stably exist at room temperature for a period of time; is less than water in density, and therefore suitable for centrifugation to generate droplets; and is heat resistant and can be subjected to a PCR cycle (up to 95° C.) without droplet fusion, therefore, it can be used for PCR reactions, and is more suitable for biological reactions with low requirement on the temperature, such as multiple displacement amplification (MDA), and loop-mediated isothermal amplification, etc. In addition to DNA and RNA reactions, droplets can also be used to separate single cells, bacteria, and proteins, etc. In addition, the oil-phase formulation of the examples of the present invention is also applicable to the method for generating droplets by a microfluidic chip, etc.

Through the dropletizing device and the centrifugal force generating device, the aqueous-phase first liquid forms small droplets under the action of the centrifugal force, and the droplets enter the second liquid located in the collecting device, to obtain the stable water-in-oil droplets, which can also be generated by a microfluidic device. The first liquid may be a sample for a biological reaction, such as a mixture for digital polymerase chain reaction, a cell suspension, a bacterial suspension, a DNA solution for genomic amplification, a mixture for RNA reverse transcription, a mixture for protein crystallization, a mixture for inorganic salt crystallization, a pathogen solution or suspension, a mixture for polymerization reaction, a mixture for gelation reaction, and the like. The second liquid is an oil-phase composition containing a surfactant. At the oil-water interface, the presence of a surfactant can stabilize the droplets against fusion. Among the above, the surfactant plays two roles: one is to form a layer of surfactant molecules with hydrophilic groups facing inward and oleophilic groups facing outward at the interface of water-in-oil droplets, forming a spatial repulsive force between droplets to prevent fusion; the other is that as the droplets move close to each other, the drainage movement of the oil phase causes a concentration gradient of the active agent molecules on the surface of the droplets, thereby generating Marangoni effect and weakening the drainage movement of the oil phase to stabilize the droplets.

Examples of the present invention provide an oil-phase composition for generating water-in-oil droplets by centrifugation, consisting of the following components: 7%-15% (v/v) of a long-chain alkyl-containing silicon-oxygen chain nonionic surfactant and 0%-10% of mineral oil, with the balance being diethylhexyl carbonate. The “long-chain alkyl” herein refers to alkyl having 10 or more carbon atoms.

The long-chain alkyl-containing silicon-oxygen chain nonionic surfactant comprises CETYL PEG/PPG-10/1 DIMETHICONE or a similar structure. For example, the long-chain alkyl-containing silicon-oxygen chain nonionic surfactant is selected from at least one of the following surfactants: ABIL® WE09, ABIL® EM180, ABIL® EM90, BC2426 (KCC Group), DIDW series from Kobo Products, Inc., and Silok® 2215, 2216 and 2216C from the Silok Chemical company.

Examples of the present invention also provide an oil-phase composition for generating water-in-oil droplets by centrifugation, consisting of the following components: 85%-95% (v/v) of a long-chain alkane ester, and 5%-15% (v/v) of a long-chain alkyl-containing silicon-oxygen chain nonionic surfactant.

The long-chain alkane ester is an alkane ester having 10 or more carbon atoms, and is selected from at least one of methyl palmitate, ethyl palmitate, isopropyl palmitate, methyl laurate, ethyl laurate, propyl laurate, isoamyl laurate, butyl laurate, methyl oleate, ethyl oleate, glyceryl oleate, methyl stearate, ethyl stearate, vinyl stearate, butyl stearate, and glyceryl stearate. For example, the long-chain alkane ester is a long-chain alkane ester having a freezing point of −10° C.-20° C., such as at least one of isopropyl palmitate, butyl laurate, methyl laurate, ethyl laurate, and butyl stearate. Under such freezing point condition, the resulting droplet oil-phase system can be stored in the refrigerator below the freezing point, for example 4° C. to greatly extend the preservation period of the sample to around one week, without the need for a stringent low-temperature condition, and when in use, it is taken out under room temperature and melted, so as to obtain a complete liquid droplet system.

The long-chain alkyl-containing silicon-oxygen chain nonionic surfactant comprises CETYL PEG/PPG-10/1 DIMETHICONE or a similar structure. For example, the long-chain alkyl-containing silicon-oxygen chain nonionic surfactant is selected from at least one of the following surfactants: ABIL® WE09, ABIL® EM180, ABIL® EM90, BC2426 (KCC Group), DIDW series from Kobo Products, Inc., and Silok® 2215, 2216 and 2216C from the Silok Chemical company.

Examples of the present invention provide an oil-phase composition for generating water-in-oil droplets by centrifugation, consisting of silicone oil and a surfactant, wherein the surfactant consists of the following substances: 5225C Formulation Aid with a content of 20%-50% (w/w), ES-5227 DM Formulation Aid with a content of about 10%-40% (w/w), ES-5612 with a content of 2%-15% (w/w), and ES-5226 DM Formulation Aid with a content of about 10%-30% (w/w). The silicone oil is 317667 silicone oil or 378321 silicone oil.

Examples of the present invention also provide a method for generating water-in-oil droplets by centrifugation using the above-mentioned oil-phase composition as a second liquid, comprising the steps of: adding to a dropletizing device a first liquid, adding to a collecting device the second liquid, and setting a rotational speed of an acceleration generating device, to generate the droplets, wherein the first liquid is in an amount of 5 μL-100 μL, the second liquid is in an amount of 300 μL-1500 μL, a vertical distance between a lower surface of the first liquid in the dropletizing device and an upper surface of the second liquid is controlled to be less than 1 cm to reduce a probability of breaking of the droplets when entering a liquid surface of the oil-phase composition, the dropletizing device comprises a porous plate for centrifugation with a pore diameter of 3 μm-10 μm and a centrifugal force of 5000 rcf-20000 rcf, which can generate uniform droplets with a diameter of 30 μm-200 μm, and the size of the droplets is mainly determined by the pore diameter and the centrifugal force of the porous plate for centrifugation.

Beneficial effects of the examples of the present invention include: 1) examples of the present invention are suitable for rapid and mass production of uniform droplets by centrifugation, and the resulting emulsion system is resistant to high temperatures and can stably exist at room temperature. In the occasion that the freezing point of the selected ester is between −10° C. and 20° C., for example, for at least one of isopropyl palmitate, butyl laurate, methyl laurate, ethyl laurate, and butyl stearate, the resulting emulsion system can be stored in a solid state below the freezing point (for example, 4° C. of the final temperature at which the PCR reaction stops) to greatly extend the preservation period of the droplets to one week, and when in use, it can be reconverted to the liquid emulsion system upon being taken out from the refrigerator under room temperature; 2) examples of the present invention are also applicable to a microfluidic device such as a microfluidic chip to form uniform and stable droplets with the reaction solution for various biological reactions; and 3) examples of the present invention can form a stable and heat-resistant emulsion system with an aqueous-phase reaction solution by a method for forming ordinary water-in-oil droplets, such as stirring, shaking, and the like. The technical solution of the present invention will be further described below by way of examples.

The first liquid is a PCR reaction solution, consisting of: 1×PCR buffer, 5 mM of MgCl₂, 0.4 mM of dNTP, and 1% of Platinum Taq Polymerase. The amount of each component is adjusted based on the commonly used combination of a double carbonate, the mineral oil and a surfactant as the oil-phase composition, to find out the ratio for the oil-phase composition that meets the requirements on density and thermal stability, and can generate a stable emulsion system with the PCR reaction solution by centrifugation. The contents of components of Examples 1-34 are as shown in Table 1.

TABLE 1
Formulations (v/v) of oil-phase compositions of Examples 1-34

| | Diethylhexyl | | |
|---|---|---|---|
| | Carbonate | ABIL WE09 | Mineral Oil |
| Example 1 | 73% | 7% | 20% |
| Example 2 | 33% | 33% | 33% |
| Example 3 | 70% | 15% | 15% |
| Example 4 | 15% | 15% | 70% |
| Example 5 | 15% | 70% | 15% |
| Example 6 | 83% | 7% | 10% |
| Example 7 | 88% | 7% | 5% |
| Example 8 | 93% | 7% | 0% |
| Example 9 | 96% | 4% | 0% |
| Example 10 | 0% | 7% | 93% |
| Example 11 | 86% | 14% | 0% |
| Example 12 | 80% | 20% | 0% |
| Example 13 | 75% | 25% | 0% |
| Example 14 | 78% | 7% | 15% |
| Example 15 | 75% | 3% | 23% |
| Example 16 | 65% | 5% | 30% |
| Example 17 | 70% | 5% | 25% |
| Example 18 | 70% | 10% | 20% |
| Example 19 | 65% | 10% | 25% |
| Example 20 | 75% | 10% | 15% |
| Example 21 | 68% | 7% | 25% |
| Example 22 | 75% | 7% | 18% |
| Example 23 | 83% | 17% | 0% |
| Example 24 | 53% | 7% | 40% |
| Example 25 | 40% | 7% | 53% |
| Example 26 | 70% | 7% | 23% |
| Example 27 | 90% | 10% | 0% |
| Example 28 | 60% | 10% | 30% |
| Example 29 | 81% | 14% | 5% |
| Example 30 | 80% | 10% | 10% |
| Example 31 | 65% | 15% | 20% |
| Example 32 | 91% | 7% | 2% |
| Example 33 | 88% | 12% | 0% |
| Example 34 | 88% | 10% | 3% |

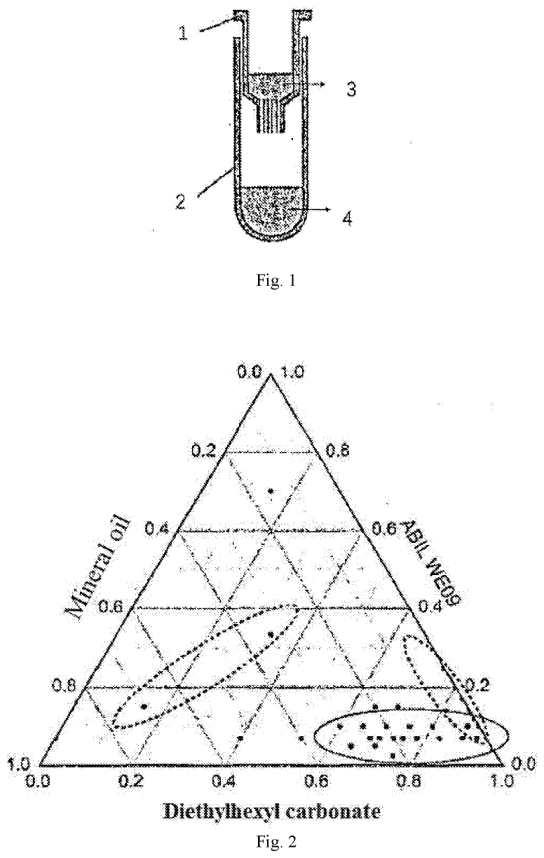

The droplet generating device (as shown in FIG. 1) in CN 104741158 A is used, wherein the reference signs are as follows: 1—dropletizing tube, 2—collecting tube, 3—first liquid, and 4—second liquid. The PCR reaction solution is used as the first liquid, the oil-phase mixture in Examples 1-34 is used as the second liquid which is located in the collecting tube 2, and the first liquid enters the second liquid under the centrifugal force to generate droplets. The second liquid, i.e., the oil-phase composition is in an amount of about 1000 μL, the first liquid, i.e., the aqueous-phase composition is in an amount of about 20 μL, and the vertical distance between the lower surface of the aqueous-phase composition and the upper surface of the oil-phase composition is controlled to be less than 1 cm to reduce the probability of breaking of the droplets when entering the liquid surface of the oil-phase composition. The porous plate for centrifugation has a pore diameter of about 6 and can generate uniform droplets with a diameter of 50 μm under a centrifugal force of 13,000 rcf. The size of the droplets is mainly determined by the pore diameter and the centrifugal force of the porous plate for centrifugation. The key factor for stable existence of droplets is the components of the oil-phase composition. It is found by extensively screening and comparing the oil-phase compositions of Examples 1-34 through a three-phase diagram (see FIG. 2) that the oil-phase compositions within the dotted circles have better thermal stability when in combination with the PCR reaction solution, the oil-phase compositions in the solid circle generate less small droplets when in combination with the PCR reaction solution, and the area where the dotted circle and the solid circle coincides, i.e., the area within which the oil-phase compositions are heat resistant and generate less small droplets, concentrates at the lower right of the figure, which corresponds to the area where the content of mineral oil is much less than that of diethylhexyl carbonate or no mineral oil is contained, and the content of ABIL WE09 is 7%-15% (v/v). The content of the surfactant ABIL WE09 cannot be too high. When it is higher than 33% (v/v), the viscosity of the oil phase is high in centrifugation, so that many small droplets are generated during entrance into the oil phase after the formation of droplets, which cannot be applied in subsequent reactions. When the content of ABIL WE09 is controlled at 15% (v/v) or less, droplets of a suitable size can be obtained. In the contrast, too low content of ABIL WE09 tends to cause droplet fusion. When it is less than 7% (v/v), the fusion is severe. Increase in the content of mineral oil will make the emulsion system tend to fuse after the high-temperature circulation, which means that the thermal stability is poor, and at the same time, the viscosity increases and the droplets are easily broken. Therefore, the content should be controlled below 10% (v/v) to ensure the integrity as well as the thermal stability of the droplets.

In order to further explore an oil-phase composition with better thermal stability, an attempt is made to find a compound similar in structure to the active agent ABILWE09 and diethylhexyl carbonate. Both ABIL® WE09 and ABIL® EMI80 are silicon-oxygen chain nonionic active agents with long-chain alkyl groups as well as similar polar group types and ratios, so they can be applied in similar systems. It is found in experiments that the active agents with the structure of CETYL PEG/PPG-10/1 DIMETHICONE or a similar structure show a better effect in stabilizing the mixed emulsion of hydrocarbon oil and water, and their HLB values are in the range of about 2-7, suitable for forming a stable water-in-oil emulsion system. In addition to ABIL® WE09 and ABIL® EM180, active agents such as ABIL® EM90, BC2426 (KCC Group), DIDW series from Kobo Products, Inc., and Silok® 2215, 2216 and 2216C from the Silok Chemical company which have the structure of CETYL PEG/PPG-10/1 DIMETHICONE or a similar structure can also be applied.

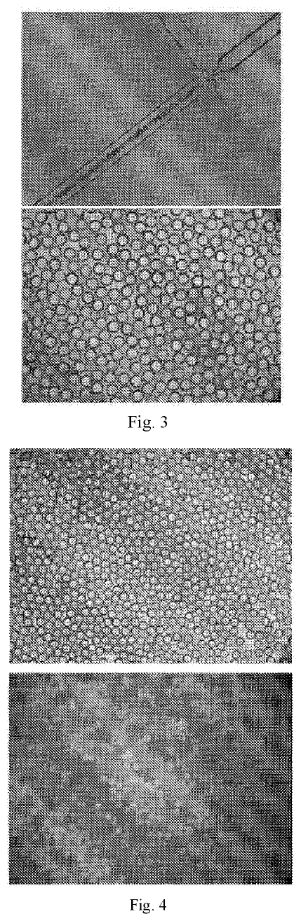

It is also found that the compositions formed by an ester with a long-chain alkyl (for example, one or more of palmitates, laurates, oleates and stearates, which have a low viscosity and a density of less than 1) and a surfactant have good results. Example 35 shows an oil-phase composition for generating water-in-oil droplets by centrifugation, consisting of 85%-95% (v/v) of isopropyl palmitate and 5%-15% (v/v) of ABIL® EM 180. The emulsion formed by the oil-phase composition and the PCR reaction solution has good stability and heat resistance. In addition, since the freezing point of isopropyl palmitate is suitable (11° C.-13° C.), the formed droplets can be stored in an oil phase solidified below 11° C. and remain stable for one week, which greatly increases the preservation period of droplets, and is beneficial to the preservation of the sample. However, by way of example, the oil phase formulation of Example 1 is difficult to be stored in a commonly-used refrigerator because its freezing point is around −30° C., and the time for the droplets to stably exist under a liquefaction condition is about 20 h. Therefore, the freezing point of the ester is preferred to be −10° C.-20° C. The ester satisfying this condition includes isopropyl palmitate, butyl laurate, methyl laurate, ethyl laurate, and butyl stearate, etc. Droplets are generated in the same manner as in Examples 1-34, and the bright-field and fluorescent photos after the PCR reaction are shown in FIG. 4. The oil-phase composition of Example 35 and the PCR reaction solution are made to pass through a Dolomite microfluidic chip (with a pore diameter of 100 μm) so as to generate droplets having a diameter of about 80 μm, as shown in FIG. 3. It can be seen that whether the oil-phase mixture is used as a second liquid for centrifugation or as a continuous phase in the generation of droplets by the microfluidic chip, the generated droplets have good performances in terms of monodispersity and high-temperature resistance.

It is found that the oil-phase composition consisting of silicone oil and a surfactant is also suitable as a second liquid for generating droplets by centrifugation. Among the above, the silicone oil is a silicone oil having a low viscosity, for example, 317667 silicone oil (viscosity 5 cSt, Sigma-Aldrich) and 378321 silicone oil (viscosity 10 cSt, Sigma-Aldrich), with a density of about 0.93 g/mL (25° C.), satisfying the density requirement in generation of droplets by centrifugation. Among the numerous silicone oil active agents, active agents of 5200 Formulation Aid, 5225C Formulation Aid, BY 11-030, ES-5612, FZ-2233, ES-5226 DM Formulation Aid and ES-5227 DM Formulation Aid suitable for the silicone oil are selected from Dow Corning® Silicone Emulsifiers for experiments. It is found in experiments that in the low-viscosity silicone oil system, the oil-phase compositions formed by 5225C Formulation Aid, ES-5612, ES-5226 DM Formulation Aid and ES-5227 DM Formulation Aid and the silicone oil have good results. The content ratio of each surfactant to silicone oil is roughly as follows: the content of 5225C Formulation Aid is 20%-50% (w/w), and the content of the silicone oil is 80%-50% (w/w); the content of ES-5227 DM Formulation Aid is about 10%-40% (w/w), and the content of the silicone oil is 90%-60% (w/w); the content of ES-5612 is 2%-15% (w/w), and the content of the silicone oil is 98%-85% (w/w); and the content of ES-5226 DM Formulation Aid is about 10%-30% (w/w), and the content of the silicone oil is 90%-70% (w/w). Droplets are generated by the same method as for Examples 1-35, which are uniform in size, can stably exist and have good thermal stability.

The above is only exemplary embodiments of the present invention, and is not intended to limit the protection scope of the present invention. The protection scope of the present invention is determined by the appended claims.

The present application claims the priority of the Chinese Patent Application No. 201610409462.8, filed on Jun. 12, 2016, the entire disclosure of which is hereby incorporated by reference as part of the present application.

1. An oil-phase composition consisting of the following components: 7%-15% (v/v) of a long-chain alkyl-containing silicon-oxygen chain nonionic surfactant, and 0%-10% (v/v) of mineral oil, with the balance being diethylhexyl carbonate.

2. The oil-phase composition of claim 1, wherein the long-chain alkyl-containing silicon-oxygen chain nonionic surfactant comprises cetyl polyethylene glycol/polypropylene glycol-10/1 dimethicone (CETYL PEG/PPG-10/1 DIMETHICONE) or a similar structure.

3. The oil-phase composition of claim 1, wherein the long-chain alkyl-containing silicon-oxygen chain nonionic surfactant is selected from at least one of the following surfactants: ABIL® WE09, ABIL® EM180, ABIL® EM90, BC2426 (KCC Group), DIDW series from Kobo Products, Inc., and Silok® 2215, 2216 and 2216C from the Silok Chemical company.

4. An oil-phase composition consisting of the following components: 85%-95% (v/v) of a long-chain alkane ester, and 5%-15% (v/v) of a long-chain alkyl-containing silicon-oxygen chain nonionic surfactant.

5. The oil-phase composition of claim 4, wherein the long-chain alkane ester has 10 or more carbon atoms.

6. The oil-phase composition of claim 4, wherein the long-chain alkane ester has a freezing point of −10° C.-20° C.

7. The oil-phase composition of claim 6, wherein the long-chain alkane ester is at least one of isopropyl palmitate, butyl laurate, methyl laurate, ethyl laurate, and butyl stearate.

8. The oil-phase composition of claim 4, wherein the long-chain alkane ester is selected from at least one of methyl palmitate, ethyl palmitate, isopropyl palmitate, methyl laurate, ethyl laurate, propyl laurate, isoamyl laurate, butyl laurate, methyl oleate, ethyl oleate, glyceryl oleate, methyl stearate, ethyl stearate, vinyl stearate, butyl stearate, and glyceryl stearate.

9. The oil-phase composition of claim 4, wherein the long-chain alkyl-containing silicon-oxygen chain nonionic surfactant comprises CETYL PEG/PPG-10/1 DIMETHICONE or a similar structure.

10. The oil-phase composition of claim 4, wherein the long-chain alkyl-containing silicon-oxygen chain nonionic surfactant is selected from at least one of the following surfactants: ABIL® WE09, ABIL® EM180, ABIL® EM90, BC2426 (KCC Group), DIDW series from Kobo Products, Inc., and Silok® 2215, 2216 and 2216C from the Silok Chemical company.

11. An oil-phase composition consisting of silicone oil and a surfactant, wherein the surfactant is selected from at least one of 5225C Formulation Aid, ES-5227 DM Formulation Aid, and ES-5612 and ES-5226 DM Formulation Aid.

12. The oil-phase composition of claim 11, wherein the silicone oil is 317667 silicone oil or 378321 silicone oil.

13. The oil-phase composition of claim 11, wherein, when the surfactant is 5225C Formulation Aid, the content of the surfactant is 20%-50% (w/w), and the content of the silicone oil is 80%-50% (w/w); when the surfactant is ES-5227 DM Formulation Aid, the content of the surfactant is 10%-40% (w/w), and the content of the silicone oil is 90%-60% (w/w); when the surfactant is ES-5612, the content of the surfactant is 2%-15% (w/w), and the content of the silicone oil is 98%-85% (w/w); and when the surfactant is ES-5226 DM Formulation Aid, the content of the surfactant is 10%-30% (w/w), and the content of the silicone oil is 90%-70% (w/w).

14. A method for generating water-in-oil droplets by centrifugation using the oil-phase composition of claim 1 as a second liquid, comprising the steps of: adding to a dropletizing device a first liquid, adding to a collecting device the second liquid, and setting a rotational speed of an acceleration generating device, to generate the droplets, wherein the first liquid is in an amount of 5 μL-100 μL, the second liquid is in an amount of 300 μL-1500 μL, a vertical distance between a lower surface of the first liquid in the dropletizing device and an upper surface of the second liquid is controlled to be less than 1 cm to reduce a probability of breaking of the droplets when entering a liquid surface of the oil-phase composition, the dropletizing device comprises a porous plate for centrifugation with a pore diameter of 3 μm-10 μm and a centrifugal force of 5000 rcf-20000 rcf, which can generate uniform droplets with a diameter of 30 μm-200 μm, and the size of the droplets is mainly determined by the pore diameter and the centrifugal force of the porous plate for centrifugation.
